(19) United States
(12) Patent Application Publication
(10) Pub. No.: US 2008/0208176 A1
(43) Pub. Date: Aug. 28, 2008

(54) INSTRUMENT FOR INJECTING AN OPHTHALMIC DEVICE INTO AN EYE

(76) Inventor: Ih-Houng Loh, Lexington, MA (US)

Correspondence Address: OCCHIUTI ROHLICEK & TSAO, LLP, 10 FAWCETT STREET, CAMBRIDGE, MA, 02138, US

(21) Appl. No.: 12/037,279

(22) Filed: Feb. 26, 2008

Related U.S. Application Data
(60) Provisional application No. 60/891,821, filed on Feb. 27, 2007.

(51) Int. Cl. A61F 9/007

(52) U.S. Cl. 606/4; 606/107

(57) ABSTRACT

An instrument for injecting an ophthalmic device such an intraocular lens into an eye includes an elongated tubular member, a plunger arranged movably along a length of and within the elongated member, a housing which accommodates an ophthalmic device, and a light emitting member integrated within the instrument. The plunger is configured to move an ophthalmic device to a distal end of the elongated member. The light emitting member illuminates a distal end of the instrument, the operative field immediately adjacent a distal end of the instrument, or both.

### CROSS-REFERENCE TO RELATED APPLICATIONS

The present invention claims the benefit of U.S. Provisional Application No. 60/891,821, filed on Feb. 27, 2007. The disclosure of this provisional application, including specification, claims, and figures, is incorporated by reference herein.

### BACKGROUND

This description relates to instruments for injecting ophthalmic devices into a patient's eye.

In surgical procedures for treatment of eye disease such as glaucoma or of age related changes to the eye such as refractive surgery to correct optical power, instruments are used to inject ophthalmic devices into the eye. Such ophthalmic devices include, but are not limited to, intraocular lenses, implantable contact lenses, capsular tension rings, and glaucoma drainage implants such as shunts or valves.

For example, an intraocular lens is an implanted lens in the eye, usually replacing the existing crystalline lens because it has been clouded over by a cataract, or as a form of refractive surgery to change the eye's optical power. The intraocular lens usually consists of a small plastic lens. The intraocular lens can be made of soft and pliable materials, wherein the intraocular lens can be folded or otherwise compacted when it is injected, i.e., implanted, in the eye through a small incision in the eye. An injection instrument is used to inject the intraocular lens into the small incision, and includes a housing, e.g., a cartridge, that accommodates the folded intraocular lens, and an actuator, e.g., a plunger, for injecting the intraocular lens into the eye.

Other ophthalmic devices such as implantable contact lenses, capsular tension rings, shunts or valves are similarly implanted in the eye through a small incision using the injection instrument. Such instruments for injecting ophthalmic devices into an eye are known in the art. For example, instruments for injection of intraocular lens include the HydroShooter™ and MecaShooter™ devices manufactured by Ophthalmic & Orthopaedic Medical Devices Consultant, Ltd., of Brighton & Hove, England, and the Viscoject™ device manufactured by Medicel AG, of Widnau, Switzerland.

Although injection instruments are frequently used with good results, there are certain problems associated with their use. For example, the intraocular device can become caught or pinched when folded into the housing or during injection into the eye. This results in a broken or torn device that does not function properly, and such a device is required to be extracted and replaced. In another example, during and after completion of the injection process, it is difficult to visually determine that the device has been properly inserted within the eye. Thus a need exists for an injection instrument which delivers an ophthalmic device into an eye through a small incision, and permits improved visual monitoring and evaluation of the injection procedure.

### SUMMARY

In one aspect, an instrument for injecting an ophthalmic device into an eye includes an elongated body member, a plunger arranged movably along a length of and within the body member and configured to move an ophthalmic device to a distal end of the body member, and a light-emitting device integrated within the instrument. The instrument also includes an insertion member engageable with the elongated body member, a portion of the insertion member configured to be inserted into the eye. The insertion member is movable along the length of and within the body member, wherein the portion of the insertion member configured to be inserted into the eye extends through an open end of the elongated member.

In some aspects, the light-emitting device is integrated within the body member. In further aspects, the light-emitting device is integrated within the insertion member. In still further aspects, light-emitting device is integrated within the plunger.

The light-emitting device is configured to illuminate an area outside the instrument. In some aspects, the area outside the instrument is illuminated through an aperture of the insertion member. In some aspects, the area outside the instrument is illuminated through an aperture of the elonagated body member. In some aspects, the insertion member is translucent, and the area outside the instrument is illuminated by light transmitted through the insertion member, the elongated body member, the plunger or all components.

The insertion member includes a housing for accommodating an ophthalmic device. The housing is configured to accommodate an ophthalmic device, and includes opposing first and second apertures. The first and second apertures are in communication along a channel formed within a length of the housing, and in use, the ophthalmic device is disposed in the channel in a folded configuration. The plunger is engageable with the housing in that a portion of the plunger is inserted through the first and second apertures and the channel, whereby the ophthalmic device is displaced along the channel and through an aperture of the insertion tip into the eye.

In one aspect, a method for injecting an ophthalmic device into an eye includes inserting a portion of an ophthalmic device injection tool into an eye, activating a light-emitting device integrated within the injection tool, and injecting an ophthalmic device from the injection tool into the eye based on a light emitted from the light-emitting device.

Implementations include one or more of the following: The method further includes controlling a movement of the ophthalmic device injection tool within the eye based on the light emitted from the light-emitting device to an area within the eye outside the injection tool. The method further includes extracting the injection tool from the eye based on the light emitted from the light-emitting device. The method further includes detecting an error in the injecting of the ophthalmic device based on the light emitted from the light-emitting device.

In one aspect, a method for assembling an instrument for injecting an ophthalmic device into an eye includes providing an elongated member, mounting a plunger arranged movably along a length of and within the elongated member for moving an ophthalmic device to a distal end of the elongated member, and integrating a light-emitting device within the instrument.

Implementations include one or more of the following: The method further includes mounting an insertion member arranged movably along a length of and within the elongated member, a portion of the insertion member configured to be inserted into the eye. Integrating the light-emitting device includes integrating the light-emitting device within the elongated member. Integrating the light-emitting device includes integrating the light-emitting device within the insertion member. Integrating the light-emitting device includes integrating the light-emitting device within the plunger.

### BRIEF DESCRIPTION OF DRAWINGS

FIG. 1 is a perspective view of an instrument for injecting an ophthalmic device into an eye.

FIG. 2 is a perspective view of a housing in an unfolded configuration.

FIG. 3 is a perspective view of the housing of FIG. 2 in a folded configuration.

FIG. 4. is a partial sectional view of the instrument of FIG. 1.

FIG. 5. is sectional view of a plunger including a fiber optic bundle.

FIG. 6 is a sectional view of a plunger including a light source and an electrical conductor.

FIG. 7 is a partial sectional view of an instrument body including a fiber optic bundle.

FIG. 8 is a partial sectional view of an instrument body including a light source and an electrical conductor.

FIG. 9 is a partial sectional view of an alternative embodiment of an instrument body including a fiber optic bundle.

FIG. 10 is a partial sectional view of an alternative embodiment of an instrument body including a light source and an electrical conductor.

FIG. 11 is a perspective view of an alternative embodiment of a housing.

FIG. 12 is a perspective view of an instrument for injecting an ophthalmic device into an eye including the housing of FIG. 11.

FIG. 13 is a sectional view of another alternative embodiment of an instrument body including light source, power supply, and switch.

FIG. 14 is a sectional view of still another alternative embodiment of an instrument body including light source, power supply, and switch.

FIG. 15 is a sectional view of a plunger including a light source and a power supply.

### DETAILED DESCRIPTION

Selected illustrative embodiments of the present invention will now be described with reference to the figures. It should be understood that only structures considered necessary for clarifying the present invention are described herein. Other conventional structures, and those of ancillary and auxiliary components of the system are assumed to be known and understood by those skilled in the art.

FIG. 1 shows an example of an instrument 10 for injecting an ophthalmic device into an eye. The instrument 10 is not limited to use in humans, and is equally well suited for use in injecting ophthalmic devices into an eye of an animal. The instrument 10 includes an elongated body 12 and a plunger 20. In some implementations, a removable housing 40 is provided that accommodates an ophthalmic device (not shown) and facilitates loading of the ophthalmic device into the elongated body 12. Ophthalmic devices include, but are not limited to, intraocular lenses, implantable contact lenses, intracorneal rings such as capsular tension rings, and glaucoma drainage implants such as shunts or valves, and may be accommodated within the housing 40 in a folded configuration.

In such implementations, the housing 40 can be arranged to be received within the elongated body 12. However, it is within the scope of the invention to provide housing that is integrated within the elongated body 12. It is further within the scope of this invention to load an ophthalmic device into the elongated body 12 by means other than a removable housing.

In the example of FIG. 1, the elongated body 12 is shown as a hollow cylindrical tube configured to accept the plunger 20 and the housing 40. In particular, the opposed ends 30, 32 of the elongated body 12 are open. The elongated body 12 also includes a pair of opposed, radially extending tab members 14, which are engaged by the fingers of a user during operation of the instrument 10. The tab members 14 are disposed adjacent the proximal end 30 of the elongated body 12. In addition, a slot 36 is formed in the distal end 32 of the elongate body 12. The slot 36 extends axially inward along the length of the elongate body, and includes a circumferentially extending protrusion 38. The slot 36 is sized to receive the housing 40 therein. In use, the housing 40 is inserted in the slot 36 so as to move along the longitudinal axis of the elongated body 12, and the protrusion 38 serves to maintain the housing 40 in the slot 36.

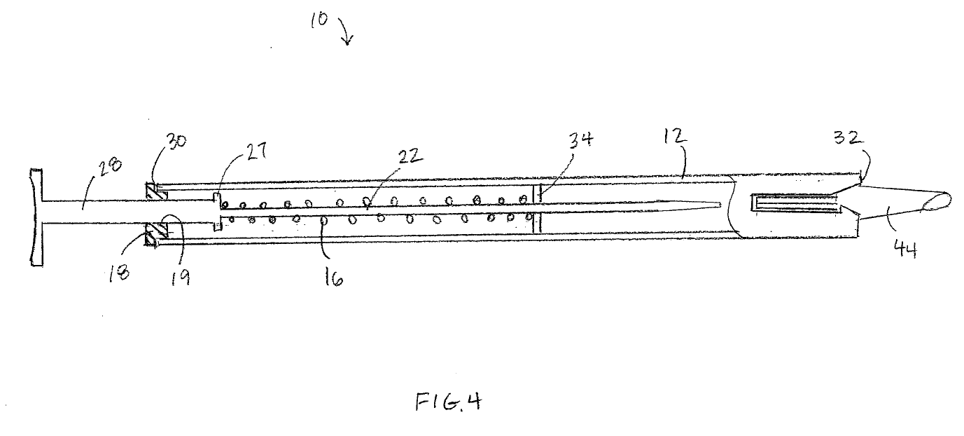

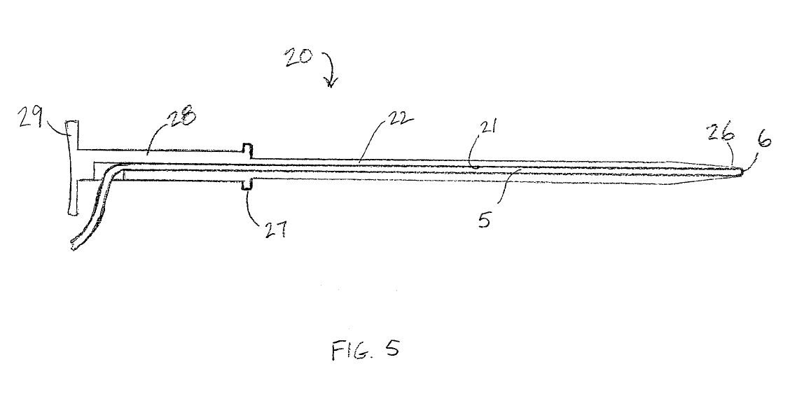

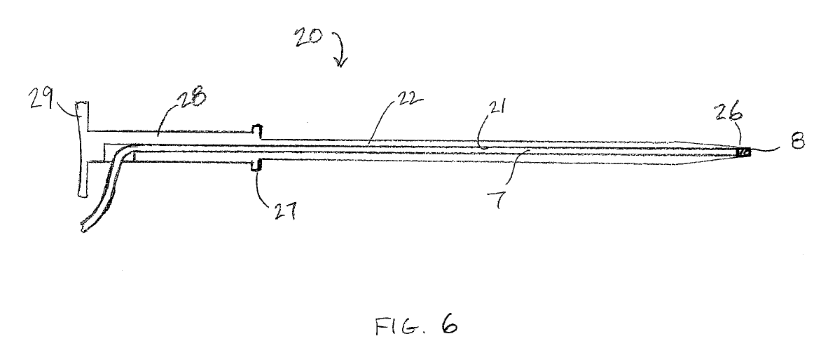

The plunger 20 (FIGS. 4 and 5) includes a rod portion 22 extending from a base portion 28, and is disposed in the proximal end 30 of the elongated body 12 so that the rod portion 22 and at least a portion of the base portion 28 are disposed within the interior of the elongated body 12. Specifically, an end cap 18 fitted within the proximal end 30 of the elongated body 12 is provided with a through hole 19, and the base portion 28 of the plunger 20 extends through, and is supported by the through hole 19 so as to permit sliding of the plunger 20 along the longitudinal axis of the elongated body 12. An annular baffle 34 (FIG. 5) is provided in a mid portion of the interior of the elongated body 12, and the rod portion 22 extends through, and is supported by the baffle 34 during movement of the plunger 20 within the elongate body 12. A resilient member 16 is disposed in a compressed state within the body 12. That is, the resilient member 16 extends between the distal end of the base portion 28 and the baffle 34. In some aspects, the resilient member 16 may be a coil spring disposed about the rod portion 22, and serves to urge the plunger 20 toward proximal end 30. A flange 27 formed at a distal end of the base portion 28 has a dimension greater than the through hole 19, whereby the plunger 20 is maintained within the elongate body 12 despite the biasing force of the resilient member 16.

The plunger 20 further includes an interior passage 21 extending axially from a proximal end of the base portion 28 and opening at the distal end face 26 of the rod portion 22. A bundle 5 of optical fibers is disposed within passage 21 such that the distal end 6 of the bundle 5 is positioned at the distal end face 26 of the rod portion. In use, an external light source (not shown) provides light to a proximal end (not shown) of the bundle 5, light is transmitted through the optical fibers of the bundle 5, and exits the distal end 6. As a result, the distal end of the plunger 20 provides a source of illumination.

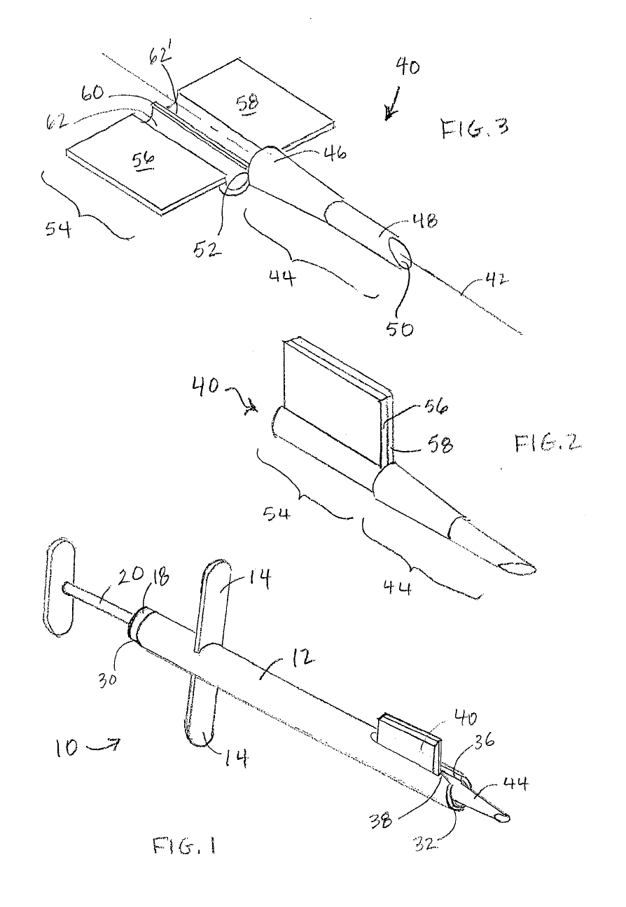

Referring now to FIGS. 2 and 3, the housing 40 includes an ophthalmic device mount portion 54 formed integrally with an insertion tip 44. The insertion tip 44 is a hollow member that is joined at a proximal end 46 to the device mount portion 54, and is tapered such that the diameter of the insertion tip 44 becomes smaller toward its distal end 48. In this regard, the tapered distal end 48 is sized so that it can be inserted into the eye through a small incision made in the eye. Openings 50, 52 at the respective distal 48 and proximal 46 ends permit communication with the hollow interior of the insertion tip 44.

The device mount portion 54 includes a first member 56 and a second member 58 which are secured or joined together and hingeably movable relative to each other along line 60, which is parallel to the longitudinal axis 42 of the housing 40. The first and second members 56, 58 are mirrored in shape, and include an arcuate portion 62, 62′ that extends along the longitudinal axis such that when the first and second members are folded together at line 60, a channel 64 is formed. When the first and second members are folded together at line 60, the channel 64 is continuous and coaxial with the hollow interior of the insertion tip 44. In use, an ophthalmic device such as an intraocular lens (not shown) is placed in the vicinity of the arcuate portions 62, 62′ of the unfolded housing, and subsequent folding of the housing 40 about line 60 results in folding of the ophthalmic device and positioning of the ophthalmic device within the channel 64.

In some implementations, the housing 40, including the insertion tip 44, can be made of a material which allows light to pass through and render any contents therein visible. Such material may be translucent, semi-transparent, or transparent. In some implementations, the elongated body 12 may also be made of a translucent, semi-transparent, or transparent material.

When the housing 40 is disposed within the slot 36, the arcuate portions 62, 62′ are received within the interior of the elongated body 12 such that the channel 64 is substantially coaxial and in communication with the interior, and such that at least the distal end 48 of the insertion tip 44 extends outwardly from distal end 32 of the elongate housing 12. When the plunger 20 is actuated by application of an axial force to the widened proximal end 29 of the base portion 28, the plunger 20 is moved within the interior of the body 12 toward its distal end 32. If a housing 40 having a folded ophthalmic device (not shown) disposed within the channel 64 resides within the slot 36 during actuation of the plunger, the distal end 26 of the rod portion 22 passes into the channel 64 and engages the ophthalmic device. Continued actuation of the plunger 20 causes the distal end 26 to drive the ophthalmic device from the channel 64, through the insertion tip 44, and outward from opening 50 at the distal end of the tip 44. The distal end 26 of the plunger 20, including the light emitting end 6 of fiber optic bundle 5, provides direct illumination of the region adjacent the distal end of the plunger 20. Thus, as the distal end 26 of the plunger 20 passes through the opening 50 of the tip 44, the region adjacent to the distal end 32 of the elongate body 12, which corresponds to a field of the incision, is provided with direct illumination.

Because the distal end 26 of the plunger 20 provides direct illumination of the field of the incision, it is possible for an operator of the injection instrument 10 to control a movement of the injection instrument 10 within the eye based on the light emitted from the plunger 20. Because the field of the incision is provided with direct illumination, it is possible to optimally extract the injection instrument 10 from the eye based on the light emitted from the light-emitting device. Moreover, because the field of the incision is provided with direct illumination, errors in the injection of the ophthalmic device and/or defects within the ophthalmic device at the time of insertion may be avoided, and if they occur, are easily detected.

In the above described embodiment, an instrument 10 for injecting an ophthalmic device into an eye includes an elongated body member 12, a plunger 20 arranged movably along a length of and within the body member 12 and configured to move an ophthalmic device to a distal end 32 of the body member 12, and a fiber optic bundle 5 integrated within the plunger 20. However, the inventive concept is not limited to this configuration. Further exemplary embodiments are now described in which like reference numbers identify like components.

In an alternative embodiment (FIG. 6), the fiber optic bundle 5 is replaced with an electrical conductor 7 which terminates in a light emitting member 8. That is, the electrical conductor 7 extends within the axial channel 21 of the plunger 20 and conducts power from and external power source (not shown) to a light emitting member 8 mounted in the distal end face 26 of the plunger 20. The light emitting member 8 can be, for example, an LED.

In another alternative embodiment (FIG. 15), a plunger 20′ is formed of light transmitting material. A light emitting member 8 and power supply 9 are disposed in a vacancy 17 formed in the widened proximal end 29 of the plunger 20′. The light emitting member 8 can be, for example, an LED. Due to the light transmitting properties of the plunger 20′, light emitted from the light emitting member 8 is transmitted through the rod portion 22 of the plunger 20′ to the distal end face 26, whereby direct illumination of housing 40, and the field of the incision in the vicinity of the distal end 32 of the injector tool 100 is provided. In some aspects, a switch 70 may be provided so as to connect the power supply 9 and the light emitting member 8. In this case, a switch arm 72 of the switch 70 protrudes from a surface of the widened proximal end 29 of the plunger 20′, such that manual engagement of the proximal end 29 actuates the switch, causing the light emitting member 8 to be switched on.

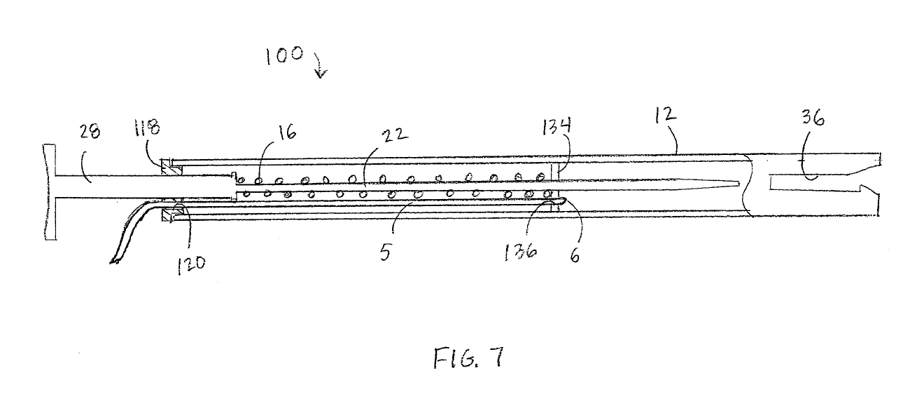

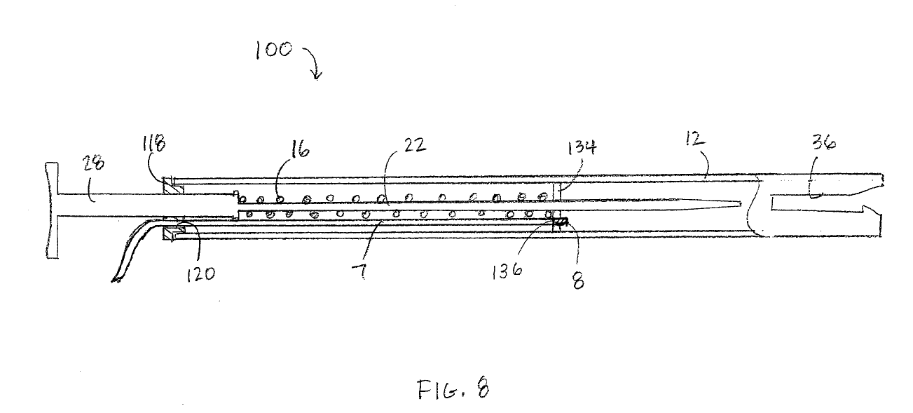

In another alternative embodiment (FIG. 7), the fiber optic bundle 5 is disposed within the elongated body 12, rather than within the plunger 20. In this embodiment, the fiber optic bundle 5 passes through an axial opening 120 formed in the end cap 118, and extends to, and is supported within an opening 136 formed in baffle 134 such that the distal end 6 of the bundle 5 is positioned to illuminate substantially the distal end half of the injector tool 100. Due to the light transmitting properties of the translucent (or transparent) elongate body 12 and housing 40, direct illumination of housing 40, and the field of the incision in the vicinity of the distal end 32 of the injector tool 100 is provided.

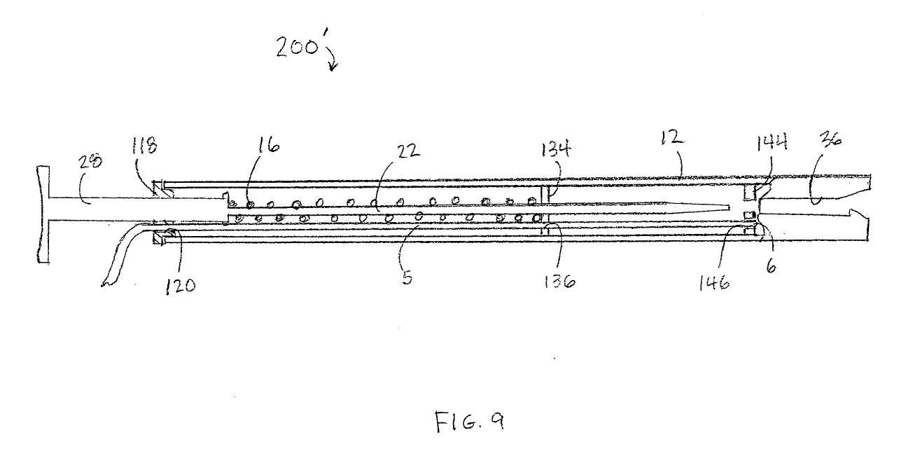

Another alternative embodiment (FIG. 8) is similar to that of FIG. 7. In the embodiment of FIG. 8, the fiber optic bundle 5 is replaced with an electrical conductor 7 which terminates in a light emitting member 8. That is, the electrical conductor 7 extends within the interior space of the elongated body 12 and conducts power from and external power source (not shown) to a light emitting member 8 mounted in the baffle 134.

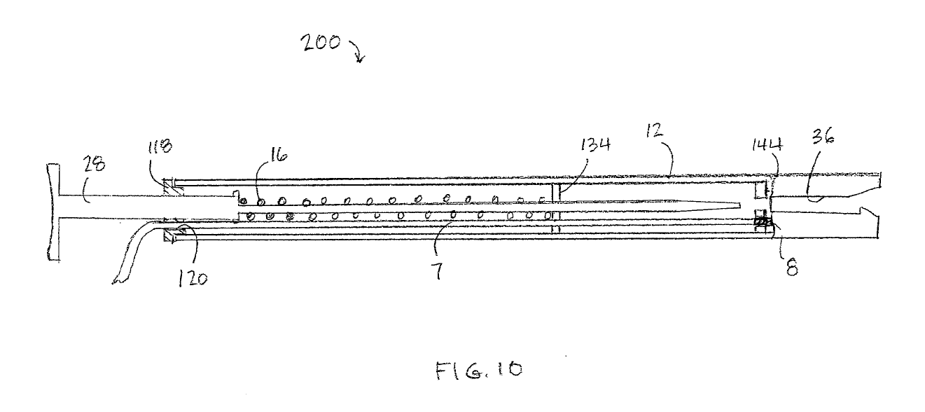

In another alternative embodiment (FIG. 9), the fiber optic bundle 5 is disposed within the elongated body 12 and terminates at a location adjacent slot 36. In this embodiment, the fiber optic bundle 5 passes through the axial opening 120 formed in the end cap 118, through the opening 136 formed in baffle 134, and extends to, and is supported within an opening 146 formed in a second annular baffle 144 such that the distal end 6 of the bundle 5 is positioned adjacent to the proximal end of the slot 36. Thus, when the housing 40 is positioned within the slot 36, the distal end 6 directly illuminates the housing 40. Due to the light transmitting properties of the translucent (or transparent) elongated body 12 and housing 40, direct illumination of the field of the incision in the vicinity of the distal end 32 of the injector tool 100 is also provided.

Another alternative embodiment (FIG. 10) is similar to that of FIG. 9. In the embodiment of FIG. 8, the fiber optic bundle 5 is replaced with an electrical conductor 7 which terminates in a light emitting member 8. That is, the electrical conductor 7 extends within the interior space of the elongated body 12 and conducts power from and external power source (not shown) to a light emitting member 8 mounted in the baffle 144.

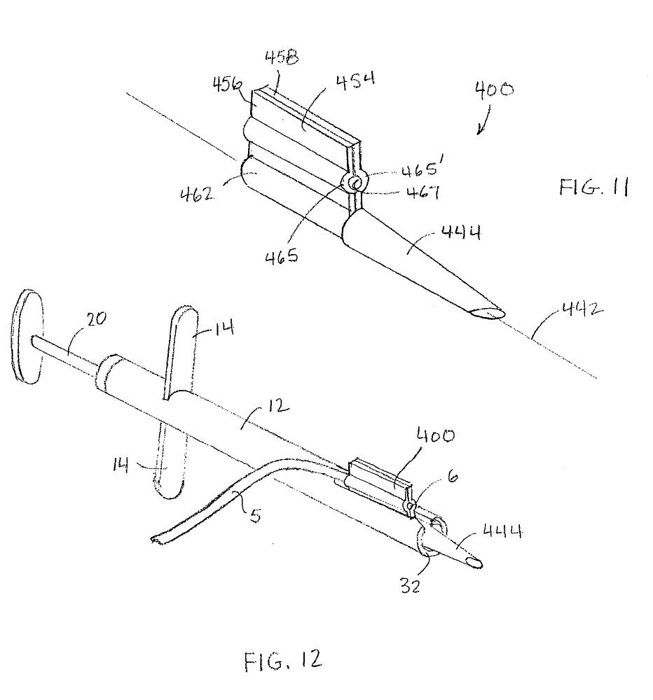

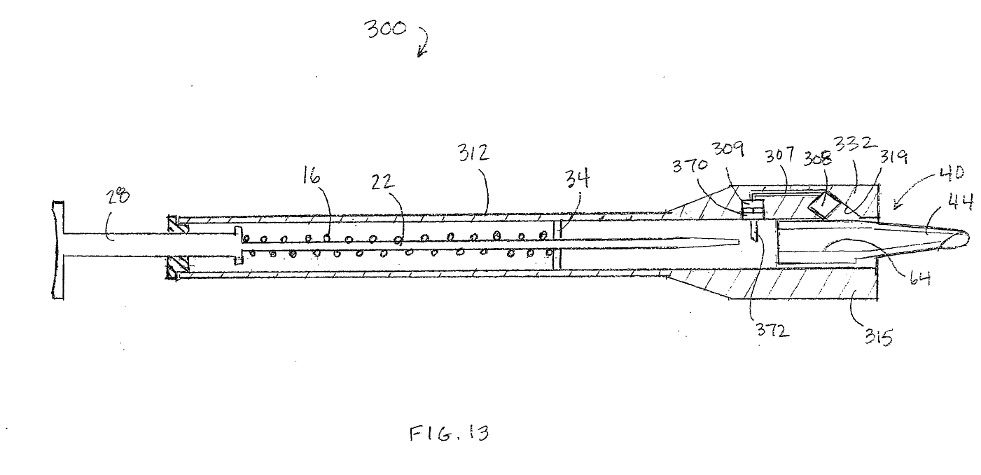

In another alternative embodiment (FIG. 12), the fiber optic bundle 5 is disposed within a modified housing 400, rather than within the plunger 20 or the elongated body 12. The housing 400 (FIG. 11) is very similar in structure to housing 40, and includes a tapered insertion tip 444 extending from a device mount portion 454. The device mount portion 454 includes a first member 456 and a second member 458 which are secured or joined together and hingeably movable relative to each other along a line (not shown) which is parallel to the longitudinal axis 442 of the housing 440. The first and second members 456, 458 are mirrored in shape, and include a first arcuate portion 462, 462′ and a second arcuate portion 465, 465′ that is slightly spaced from the first arcuate portion 462, 462′. Both arcuate portions 462, 465 extend in parallel to the longitudinal axis such that when the first and second members 456, 458 are folded together, a first channel 464 and second channel 467 are formed. The first channel is continuous and coaxial with the hollow interior of the insertion tip 444. When the housing 400 is inserted in the slot 36 of the elongated body 12, the first arcuate portion 462, 462′ is received within the interior of the elongated body 12, and the second arcuate portion 465, 465′, including channel 467, resides exteriorly of the elongate body 12 adjacent to the slot 36. In use, the fiber optic bundle 5 passes through the axial channel 467 such that the light emitting end 6 is disposed at a distal end of the channel 467 to provide illumination of the insertion tip 444 and the region adjacent to the distal end of the elongated body 12 corresponding to an operative field.

In the above described embodiments, the instruments 10, 100, 200 for injecting an ophthalmic device into an eye include a fiber optic bundle 5 in which a light source, and its power supply is located externally of the instrument, or include a light emitting member 8 which is powered externally of the instrument. It is considered that it may be convenient to provide a ophthalmic device injection instrument in which the light source and power supply are contained within the instrument.

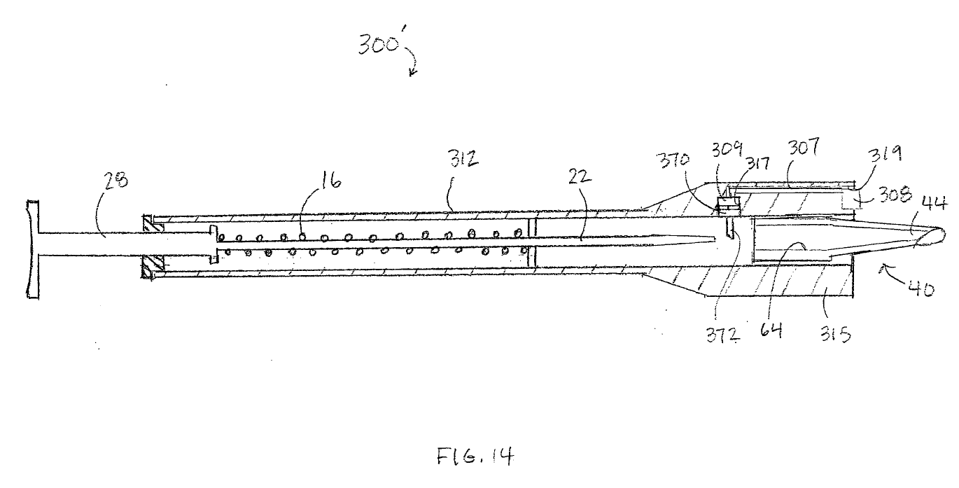

In an alternative embodiment shown in FIG. 13, an ophthalmic device injection instrument 300 includes an elongated body 312, and a plunger 20 disposed within the elongated body 312 and biased toward the proximal end of the elongated body 312 by an elastic member 16. Like previous embodiments, an ophthalmic device-containing housing 40 is received in a slot formed at the distal end 332 of the elongated body 312.

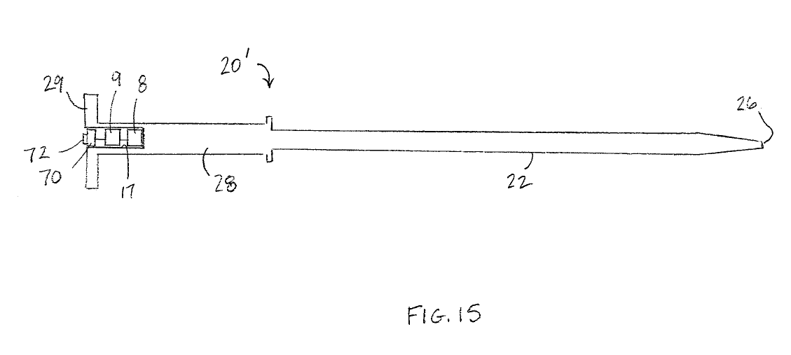

The elongated body 312 is provided with a widened portion 315 at the distal end 332 thereof. A light source 308, power supply 309 and switch 370 are mounted within the widened portion 315 so as to provide illumination of the region adjacent the distal end 332 of the elongate body 312, including the housing 40 and a field of operation external to the instrument 300. In particular, the power supply 309, embodied for example by a battery, is disposed along with the switch 370 within a first vacancy 317 formed in the widened portion 315. The vacancy 317 opens into the interior of the elongate body 312, and the switch 370 is mounted within the vacancy 317 so that a switch arm 372 of the switch 370 extends into the travel path of the rod portion 22 of the plunger 20. Thus, during activation of the plunger 20, as the rod portion 22 approaches the channel 64 of the housing 40, the switch arm 372 is displaced by the rod portion 22. As a result, the switch 370 is activated and power is supplied to the light source 308 via a conductor 307 extending between the power supply 309 and the light source 308. In the embodiment shown in FIG. 13, the light source 308 is disposed in a second vacancy 319 within the widened portion. The second vacancy 319 opens into the interior of the elongated body 312 at a location confronting the housing 40. In some embodiments, the second vacancy 319 is angled with respect to the longitudinal axis of the body 312 to direct light toward the tip 44 of the housing 40. However, the arrangement of the second vacancy 319 and the light source 308 therein is not limited to this configuration. For example, as shown in the embodiment of FIG. 14, the light source 308 may be disposed in a second vacancy 319′ within the widened portion 315, and the second vacancy 319′ is proved in the distal end face 332 of the elongated body 312 such that it opens into the exterior of the elongated body 312.

In these embodiments, the light source 308 may be an LED, conventional lamp, or other known light emitting element. The light source 308 may also include a plurality of light emitting elements, arranged to direct light on the housing 40, toward the region adjacent the distal end 332 of the instrument corresponding to an operating field, or both.

In these embodiments, the switch may be actuated by means other than the plunger. For example, a manual switch may be provided on an external surface of the elongated body 312. In addition, the switch 310, power supply 309, and light source 308 may all be housed in a single vacancy.

In the embodiments described herein, the housing 40 includes a device mount portion 54 formed integrally with the insertion tip 44. However, it is within the scope of the invention to form the device mount portion 54 for accommodating an ophthalmic device as a separate member from the insertion tip 44. In such a device, the insertion tip 44 may be formed integrally at the distal end 32 of the elongate body 12, or may be selectively engageable therewith by, for example, press fitting or use of complimentary screw threads.

In the embodiments described herein, the injection instrument may be a single-use, disposable instrument, or, alternatively, the same injection instrument may be used to achieve multiple injections. A single-use instrument may be formed of inexpensive materials such as plastics, which may include, but are not limited to, polycarbonate or polypropylene. As previously discussed, use of plastics may be also be advantageous due to their light transmittal properties. Multiple-use instruments may be formed of materials such as titanium or stainless steel.

A method for injecting an intraocular device into an eye using a light emitting ophthalmic device injection instrument as described above includes the following method steps:

Forming an incision in an eye;

Inserting at least a portion of an ophthalmic device injection tool into the incision;

Activating a light-emitting device integrated within the injection tool. This step may precede one or both of the previous method steps; and

Injecting an ophthalmic device from the injection tool into the eye based on observations of the field of the incision using light emitted from the light-emitting device.

The method may include the additional steps of controlling a movement of the ophthalmic device injection tool within the eye based on observations of the field of the incision using the light emitted from the light-emitting device to an area within the eye outside the injection tool, extracting the injection tool from the eye based on the light emitted from the light-emitting device, and detecting an error in the injecting of the ophthalmic device based on the light emitted from the light-emitting device.

A method of assembling an instrument for injecting an ophthalmic device into an eye includes the following method steps:

Providing an elongated member;

Mounting a plunger arranged movably along a length of and within the elongated member for moving an ophthalmic device to a distal end of the elongated member;

Mounting an insertion member arranged movably along a length of and within the elongated member, a portion of the insertion member configured to be inserted into the eye; and

Integrating a light-emitting device within the instrument.

The method step of integrating the light-emitting device includes integrating the light-emitting device within the elongated member, integrating the light-emitting device within the insertion member, or integrating the light-emitting device within the plunger.

While working examples of the present invention and associated methods have been described above, the present invention is not limited to the working examples described above, but various design alterations may be carried out with departing from the present invention as set forth in the claims.

What is claimed is:

1. An instrument for injecting an ophthalmic device into an eye, the instrument comprising:
an elongated member;
a plunger arranged movably along a length of and within the elongated member and configured to move an ophthalmic device to a distal end of the elongated member; and
a light-emitting device integrated within the instrument.

2. The instrument of claim 1, further comprising:
an insertion member engageable with the elongated member, a portion of the insertion member configured to be inserted into the eye.

3. The instrument of claim 1, wherein:
the light-emitting device is integrated within the elongated member.

4. The instrument of claim 2, wherein:
the light-emitting device is integrated within the insertion member.

5. The instrument of claim 1, wherein:
the light-emitting device is integrated within the plunger.

6. The instrument of claim 5, wherein at least a distal end of the instrument is illuminated via light transmitted through the plunger.

7. The instrument of claim 5, wherein
the plunger is formed of a light transmitting material;
the light emitting device is disposed at a proximal end of the plunger; and
at least a distal end of the instrument is illuminated via light transmitted through the plunger.

8. The instrument of claim 2, wherein:
the light-emitting device is configured to illuminate an area outside the instrument through an aperture of the insertion member.

9. The instrument of claim 2, wherein:
the insertion member is translucent.

10. The instrument of claim 2, wherein the insertion member comprises:
a housing for accommodating an ophthalmic device, the housing including a first aperture;
wherein the portion of the insertion member configured to be inserted into the eye includes a tip, the tip including a second aperture in communication with the first aperture via a channel formed along a length of the housing.

11. The instrument of claim 10, wherein:
the plunger is engageable with the insertion member through the first aperture to inject the ophthalmic device from the housing through the channel and the second aperture into the eye.

12. The instrument of claim 1 wherein the light emitting device comprises an LED.

13. The instrument of claim 1 wherein the light emitting device comprises a bundle of fiber optics.

14. An instrument for injecting an ophthalmic device into an eye, the instrument comprising:
an elongated member including an insertion tip, the insertion tip configured to be inserted through an incision formed in the eye;
a plunger arranged movably along a length of and within the elongated member and configured to move an ophthalmic device to a distal end of the insertion tip; and
a light-emitting device integrated within the instrument.

15. The instrument of claim 14, further comprising:
a housing arranged within the elongated member, the housing configured to accommodate the ophthalmic device.

16. The instrument of claim 14, wherein:
the light-emitting device is integrated within the elongated member.

17. The instrument of claim 14, wherein:
the light-emitting device is integrated within the elongated member at the insertion tip.

18. The instrument of claim 14, wherein:
the light-emitting device is integrated within the plunger.

19. The instrument of claim 14, wherein:
the light-emitting device is configured to illuminate an area outside the instrument through an aperture of the insertion tip.

20. The instrument of claim 14, wherein:
the insertion tip is translucent.

21. The instrument of claim 15, wherein:
the housing includes opposing first and second apertures, the first and second apertures in communication along a channel formed within a length of the housing;
wherein the plunger is engageable with the housing through the first and second apertures and the channel to inject the ophthalmic device through an aperture of the insertion tip into the eye.

22. The instrument of claim 14 wherein the light emitting device comprises an LED.

23. The instrument of claim 14 wherein the light emitting device comprises a bundle of fiber optics.

24. A method for injecting an ophthalmic device into an eye, the method comprising:
inserting at least a portion of an ophthalmic device injection tool into an eye;
activating a light-emitting device integrated within the injection tool; and
injecting an ophthalmic device from the injection tool into the eye based on a light emitted from the light-emitting device.

25. The method of claim 24, further comprising:
controlling a movement of the injection tool within the eye based on the light emitted from the light-emitting device to an area within the eye outside the injection tool.

26. The method of claim 24, further comprising:
extracting the injection tool from the eye based on the light emitted from the light-emitting device.

27. The method of claim 24, further comprising:
detecting an error in the injecting of the ophthalmic device based on the light emitted from the light-emitting device.

28. A method for assembling an instrument for injecting an ophthalmic device into an eye, the method comprising:
providing an elongated member;
mounting a plunger arranged movably along a length of and within the elongated member for moving an ophthalmic device to a distal end of the elongated member; and
integrating a light-emitting device within the instrument.

29. The method of claim 28, further comprising:
mounting an insertion member arranged movably along a length of and within the elongated member, a portion of the insertion member configured to be inserted into the eye.

30. The method of claim 29, wherein integrating the light-emitting device comprises:
integrating the light-emitting device within the insertion member.

31. The method of claim 28, wherein integrating the light-emitting device comprises:
integrating the light-emitting device within the elongated member.

32. The method of claim 28, wherein integrating the light-emitting device comprises:
integrating the light-emitting device within the plunger.

33. An instrument for injecting an ophthalmic device into an eye, the instrument comprising:
a hollow elongated member;
a housing, the housing received within the member and configured to support an ophthalmic device within the member;
a plunger disposed in an opening formed in a proximal end of the member, the plunger including a rod portion arranged to move within the elongated member and housing; and
a light-emitting device.

34. The instrument of claim 33, wherein the light-emitting device is integrated within the member.

35. The instrument of claim 33, wherein the light-emitting device is integrated within the housing.

36. The instrument of claim 33, wherein the light-emitting device is integrated within the plunger.

37. The instrument of claim 33 wherein the light emitting device comprises an LED.

38. The instrument of claim 33 wherein the light emitting device comprises a bundle of fiber optics.

39. The instrument of claim 33 wherein the light emitting device is configured to illuminate a distal end of the instrument.

40. The instrument of claim 33 wherein the light emitting device is configured to illuminate a region adjacent a distal end of the member.
